(19) United States
(12) Patent Application Publication
(10) Pub. No.: US 2013/0251740 A1
(43) Pub. Date: Sep. 26, 2013

(54) IMMUNOGENIC AGENTS

(71) Applicants: Fotini Papavasiliou, New York, NY (US); Pete Stavropoulos, New York, NY (US)

(72) Inventors: Fotini Papavasiliou, New York, NY (US); Pete Stavropoulos, New York, NY (US)

(73) Assignee: ROCKEFELLER UNIVERSITY (THE), New York, NY (US)

(21) Appl. No.: 13/881,993

(22) PCT Filed: Sep. 19, 2011
(86) PCT No.: PCT/US11/52143

§ 371 Date: Jun. 4, 2013

Related U.S. Application Data
(60) Provisional application No. 61/406,761, filed on Oct. 26, 2010.

(51) Int. Cl. C07K 14/00

(52) U.S. Cl. CPC C07K 14/00
USPC 424/191.1; 530/395; 536/23.7; 435/320.1; 435/258.1

(57) ABSTRACT

The present invention relates to immunogenic agents based on trypanosomes, related compositions, and related methods.

### CROSS REFERENCE TO RELATED APPLICATION

This application claims priority of U.S. Provisional Application No. 61/406,761 filed on Oct. 26, 2010. The content of the application is incorporated herein by reference in its entirety.

### GOVERNMENT INTERESTS

The invention disclosed herein was made, at least in part, with Government support under Grant No R01AI085973 the National Institute of Allergy and Infectious Diseases/National Institutes of Health and “Transformative” Grant No R01AI097127, from the Office of the Director/National Institutes of Health. Accordingly, the U.S. Government has certain rights in this invention.

### FIELD OF THE INVENTION

This invention relates to immunogenic agents based on trypanosomes (e.g., Trypanosoma brucei (T. brucei)), related compositions, and related methods.

### BACKGROUND OF THE INVENTION

The immune system defends the human body against pathogen infection, cellular transformation, and physical/chemical damage. Immunization enables an individual's immune system to become fortified against an agent (known as the immunogen). The basis of immunization, under which vaccination resides, is to generate protective titers of blood-borne antibody (humoral response) as well as memory B-cells (cellular response) that can protect against infectious disease. Typically, vaccines contain agents that resemble a disease causing microorganism and elicit a humoral and/or cellular immune response against the microorganism. These agents can be made of killed microbes, parts of a microbe, or its toxins. All of these immunogens, upon injection, can provide a protective response in a subject. An immunogen, thus, stimulates the body's immune system to recognize exposure to the entire pathogen upon prior exposure to the immunogen itself, and then destroy the pathogen.

Generation of a proper vaccine, which is contingent upon finding the proper immunogen that will generate a sufficient protective antibody and memory B-cell response, is a continuing challenge to vaccine development. Approaches to solving this problem have included reverse engineering though structural biology which first deciphers the structure of an antibody bound to a target protein, pinpoints the specific interaction domain between antibody and protein (called an epitope), and then uses that epitope as a designer immunogen.

Once such an immunogen is identified, in principle, one can inject it into an organism and generate the proper antibody response. However, this technique has proven very challenging in practice. While the proper antibody can be used to identify the proper epitope, injection of the proper epitope does not always generate the correct antibody. The reasons include: tolerance, the mechanism which usually protects a subject from making antibodies against human proteins; the poor immunogenicity of key epitopes; the problem that conformational epitopes (i.e., epitopes that may be in proximity in three dimensions but could belong to different parts of the protein when it is in linear form) are particularly difficult to construct, and finally, the inability to stimulate a memory response. General solutions to these problems do not exist, with the exception of increasing the immunogenicity of key epitopes. This can be achieved by simply increasing the number of epitopes exposed to the immune system. Methods to increase the number of epitopes exposed to the immune system include inserting them into pseudotyped viruses or special nanomaterials designed platforms. However, success of these platforms remains inconsistent.

Therefore, there remains a continuing unmet need to develop a vaccine vector which can reliably provide sufficiently well displayed immunogenic epitopes and can elicit a protective immune response in a subject.

### SUMMARY OF INVENTION

This invention relates to agents based on the African trypanosome (e.g., T. brucei), including chimeric proteins and related immunogenic compositions (e.g., vaccines), and related methods to generate an immune response, as well as a protective or therapeutic immune response in a subject (human or animal).

One aspect of this invention features a chimeric protein having (i) one or more alpha helices or beta-strands of a Trypanosoma brucei variable surface glycoprotein (VSG protein) or a fragment thereof and (ii) a first heterologous polypeptide sequence. The chimeric protein can further contain a second heterologous polypeptide sequence. The first or second heterologous polypeptide sequence can be 1-50 (i.e., any integer between 1 and 50, inclusive, e.g., 2, 3, 5, 10, 15, 20, 25, 30, 40, and 50) amino acids in length. The first and second heterologous polypeptide sequences can be the same or be different.

In certain embodiments, the first or second heterologous polypeptide sequence contains an epitope of a protein selected from the group consisting of a pathogen protein, a tumor antigen, an epitope associated with conformational disorders (e.g. amyloid beta, Tau, etc), or the prion protein (PrP^{C}). In one example, the first or second heterologous polypeptide sequence contains DYKDDDDK (FLAG epitope, SEQ ID NO: 2) or YPYDVPDYA (HA epitope, SEQ ID NO: 3). In another example, the VSG protein that can be used for this invention contains the sequence of the N-terminal domain of VSG427-2 (SEQ ID NO: 1 shown in FIG. 1A). In yet another example, the chimeric protein contains a mutant version of SEQ ID NO: 1 where the first or second heterologous polypeptide sequence is inserted at one or more of positions 28, 144, 145, 153, 167, 203, 221, 227, 247, 249, 259, 262, 263, and 298.

One can generate the aforementioned chimeric protein by (1) introducing an expression vector encoding the protein into a host cell, (2) culturing the host cell in a medium under conditions permitting expression of the polypeptide, and (3) purifying the polypeptide. A vector refers to a nucleic acid molecule capable of transporting another nucleic acid to which it has been linked. The vector can be capable of autonomous replication or integrate into a host DNA. Examples of vectors include a plasmid, cosmid, or viral vector. The vector of this invention includes a nucleic acid in a form suitable for expression of the nucleic acid in a host cell. Preferably the vector includes one or more regulatory sequences operatively linked to the nucleic acid sequence to be expressed. The term regulatory sequence includes promoters, enhancers, and other expression control elements (e.g., splicing signals, untranslated regions, polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of transcription of RNA desired, and the like.

In this application, the above-mentioned host cell is a Trypanosoma brucei cell. Thus, the invention also features a recombinant Trypanosoma brucei containing the above-mentioned chimeric protein or nucleic acid.

In a second aspect, the invention features a conjugate having a Trypanosoma brucei variable surface glycoprotein (VSG protein) and a heterologous moiety, wherein the heterologous moiety is conjugated to the VSG protein. The heterologous moiety can be a large polypeptide or a small molecule hapten. The polypeptide can contain an epitope of a protein selected from the group consisting of a pathogen protein, a tumor antigen, an amyloid beta, a Tau peptide, and a prion protein (PrP^{C}), or any other protein of interest against which the generation of antibodies is desired. The small molecule hapten can be a model hapten such as the commonly used immune stimulators nitrophenyl (NP), or, can be a small molecule which is a drug of abuse such as nicotine, methamphetamine, morphine and derivatives thereof (e.g. diacetylmorphine (also known as heroin), or cocaine and derivatives thereof.

In a third aspect, the invention features an immunogenic composition containing (i) a pharmaceutically acceptable small molecule and (ii) one or more of the above-mentioned chimeric proteins, the recombinant Trypanosome brucei, or conjugates.

In a fourth aspect, the invention features a method of producing antibodies that recognize a polypeptide in a subject, or of eliciting an antigen-specific immune response in a subject. The method includes a step of administering to the subject the just-mentioned immunogenic composition. The method can further include a step of obtaining antibodies or a sample containing the antibodies from the subject.

The details of one or more embodiments of the invention are set forth in the description below. Other features, objects, and advantages of the invention will be apparent from the description and from the claims.

### BRIEF DESCRIPTION OF THE DRAWINGS

FIGS. 1a-d are diagrams showing: (a) a number of linear epitopes inserted at multiple positions within VSG loops that can be displayed on the surface of T. brucei., where the structure of the N-terminal domain of VSG427-2 is shown on the left; secondary structure elements are presented above the primary sequence of VSG427-2 where the shaded rectangles represent alpha-helices and the arrows represent beta-strands; the loops connecting the secondary structural elements are depicted in various colors that correlate the location of the loop on the structure with the secondary structure/primary sequence alignment. The location and sequence of the epitope insertions are presented within the single horizontal bracket above the sequence of VSG427-2 followed by a number which represents the transgenic trypanosome that expresses the chimeric/fusion VSG with the epitope in the indicated position; (b) flow cytometry analysis of transgenic VSG427-2 strains with chimeric/fusion surface expression of FLAG epitope where the strains were stained with an anti-FLAG antibody conjugated to fluorescein (anti-FLAG-FITC); (c) flow cytometry analysis of un-transfected strain expressing VSG427-2 as assessed by surface staining with an antibody against 427-2 labeled with FITC, also known as an “anti-221” antibody; (d) flow cytometry analysis of cells transfected to express HA peptides the surface expression of which is assessed by staining with an anti-HA antibody conjugated to phycoerythrin (PE).

FIGS. 2a-c are diagrams showing that inoculation of mice with chimeric trypanosomes elicited specific antibodies against FLAG (a) and HA (b) at various times points as measured by standard ELISA and protected the mice against T. brucei (c).

FIGS. 3a-e are diagrams showing insertion of a conserved epitope of the influenza hemagglutinin protein into VSG (a and b), FACS results showing surface expression of said epitope (c), Western blots showing that anti-influenza antisera from the injected mice recognize influenza hemagglutinin protein (d) and prophetic examples of protection after influenza infection (e).

FIGS. 4a-f are diagrams showing insertion of a conserved epitope of the prion protein (Prp) into VSG (a and b), FACS results showing surface expression of the epitope (c), Western blots showing that anti-PrP antisera from the injected mice recognize full length PrP protein, (d) FACS experiments showing that anti-PrP antisera from the injected mice recognize PrP protein on the surface of B cells (e)—which is a recognized measure of their ability to be protective and (f) prion disease ablation after scrapie injection (f).

FIGS. 5a-d are diagrams showing insertion of epitopes of RD4 and 164 of AID protein into VSG to generate chimeric proteins and trypanosomes expressing the chimeric proteins, which elicited antibodies to the epitopes in mice.

### DETAILED DESCRIPTION OF THE INVENTION

This invention is based, at least in part, on unexpected discoveries that a trypanosome (e.g., T. brucei) variable surface glycoprotein (VSG) protein is tolerant to exogenous peptide insertions in its exposed loop regions, and can display exogenous peptides with high efficiency on the surface of a trypanosome cell.

### Trypanosomes

Trypanosome is a genus of kinetoplastids (class Kinetoplastida), a monophyletic group of unicellular parasitic flagellate protozoa. Trypanosomes infect a variety of hosts and cause various diseases, including the fatal human diseases sleeping sickness, caused by a subspecies of T. brucei (T. brucei rhodensiensis), and Chagas disease, caused by T. cruzi.

T. brucei (henceforth simply T. brucei), the African trypanosome used throughout this application, is a cattle (though not human) pathogen, transmitted by the bite of the tsetse vector to the mammalian bloodstream. T. brucei exists in a completely extracellular form in the mammalian bloodstream and is constantly exposed to the immune system. As a result of this exposure, the parasite elicits a robust immune response that is almost exclusively antibody mediated, and is extremely specific to the trypanosome surface.

The surface of T. brucei consists of about 11 million copies of a VSG protein. Each VSG is anchored to the cell membrane via a glycophosphatidylinositol (GPI) anchor—a covalent linkage from the C terminus, to approximately four sugars, to a phosphatidylinositol phospholipid acid which lies in the cell membrane. The crystal structure of VSG221 reveals a very well structured protein that is packed tightly on the surface of the parasite, with a limited area composed of structured loops exposed on the top. They present a uniform repetitive surface to the immune system; thus, eliciting robust antibody responses (Overath et al., 1994, Parasitol Today 10, 53-8). This dense and repetitive protein coat naturally stimulates potent and specific antibody responses, as well as B cell memory. As a result of the robust antibody response, during infection of its mammalian host, trypanosomes are removed from the bloodstream through specific antibody binding (Borst, 2002, Cell 109, 5-8). These loops are well defined in the structure (Blum et al., 1993, Nature 362, 603-9) and are resistant to proteolytic cleavage indicating that they contribute to the overall structural integrity and function of this molecule. It would be expected that removal of these loops, even a small portion of them, would very well lead to destabilization of the overall protein structure.

As disclosed herein, trypanosomes can be a powerful platform for the immunogenic display of antigenic determinants toward the generation of antibodies. Accordingly, this invention provides systems and methods to display repetitive, ordered arrays of linear and/or conformational epitopes on the surface of T. brucei. The engineered chimeric proteins and organisms can generate specific anti-epitope antibody responses, upon injection into a subject (such as a mouse, rabbit, camel, or chicken, or other suitable animal). The invention disclosed herein offers an alternative approach to generating antibodies, and can be used to generate protective vaccines.

### Chimeric Protein

This invention provides an isolated or purified chimeric trypanosome VSG protein. A chimeric protein or fusion protein as used herein in refers to a protein created through the joining of two or more heterologous proteins or polypeptides.

A “chimeric” or “fusion” refers to the combination of amino acid sequences of different origin in one polypeptide chain by in-frame combination of their coding nucleotide sequences. The term explicitly encompasses internal fusions, i.e., insertion of sequences of different origin within a polypeptide chain, in addition to fusion to one of its termini.

A heterologous protein, polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

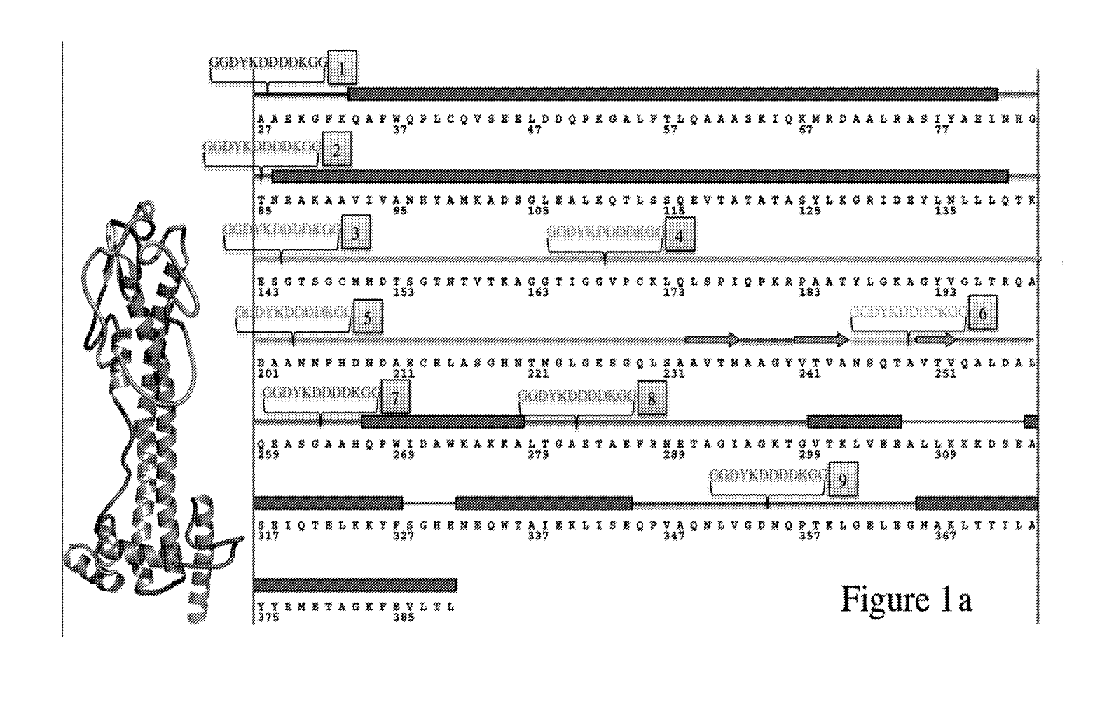

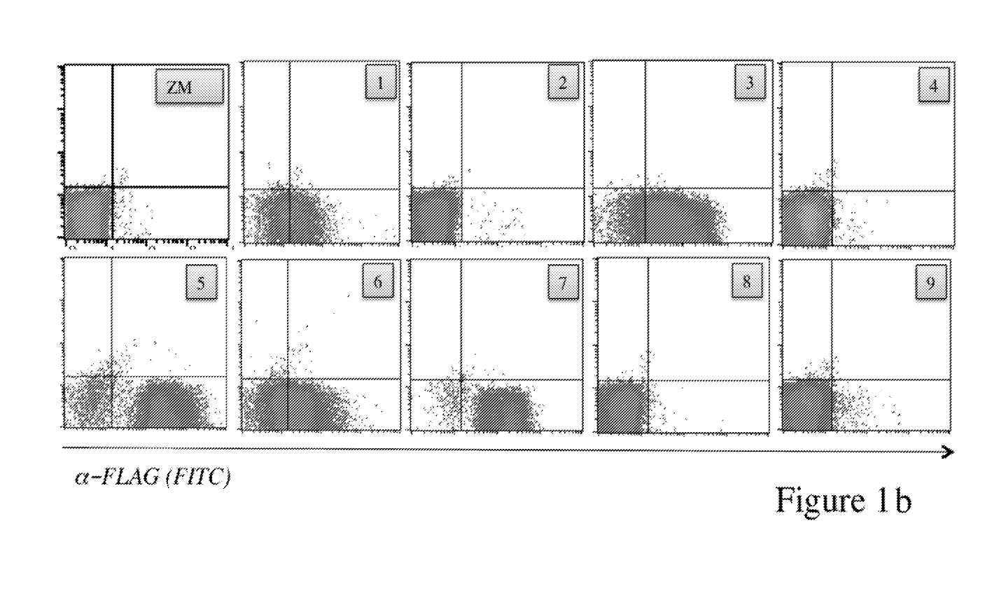

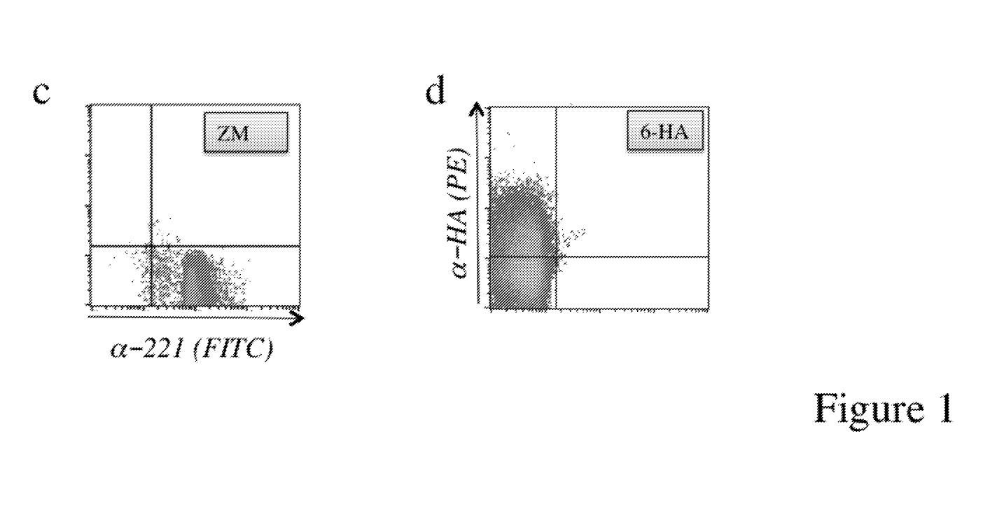

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation). The peptide, polypeptide, or protein “of this invention” includes recombinantly or synthetically produced fusion versions having the particular domains or portions of VSG, e.g., one or more alpha helices or beta-strands shown in FIG. 1. The term also encompasses polypeptides that have an added amino-terminal methionine (useful for expression in prokaryotic cells).

An “isolated” or “purified” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein described in the invention can be purified from a natural source, produced by recombinant DNA techniques, or by chemical methods.

As mentioned above, the surface of the trypanosome is covered by a dense coat of VSG proteins, which allows persistence of an infecting trypanosome population in the host. The dense nature of the VSG coat prevents the immune system of the infected mammalian host from accessing the plasma membrane or any other invariant surface epitopes of the parasite. The coat is uniform, but monoallelic, that is, it is made up of millions of copies of the same molecule; therefore the only parts of the trypanosome the immune system can see are loops within the N-domain of the VSG.

VSG genes are variable at the sequence level. However, for them to fulfill their shielding function, different VSGs have strongly conserved structural features. VSGs are made up of a highly variable N terminal domain of around 300 to 350 amino acids, and a more conserved C terminal domain of around 100 amino acids. Shown on FIG. 1a are the structure of the N-terminal domain of VSG427-2 (on the left) and the corresponding primary sequence with secondary structure elements shown: the shaded rectangles represent seven alpha-helices and the arrows represent beta-strands; the structured loops connecting the secondary structural elements are depicted as well.

A chimeric trypanosome VSG protein as disclosed herein is a fusion protein having one or more alpha helices or beta-strands of a trypanosome VSG protein (e.g., VSG protein of T. brucei) and one or more heterologous polypeptide sequences. The heterologous polypeptide sequences are located in the structured loop regions of the VSG.

A protein described in this invention can be obtained as a recombinant protein. To prepare a recombinant protein, a nucleic acid encoding it can be linked to another nucleic acid encoding a fusion partner, e.g., VSG, glutathione-s-transferase (GST), 6×-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention. Alternatively, the peptides/polypeptides/proteins of the invention can be chemically synthesized (see e.g., Creighton, Proteins: Structures and Molecular Principles, W.H. Freeman & Co., NY, 1983). For additional guidance, skilled artisans may consult Ausubel et al. (supra), Sambrook et al. (Molecular Cloning, A Laboratory Manual, Cold Spring Harbor Press, Cold Spring Harbor, N.Y., 1989).

A “recombinant” peptide, polypeptide, or protein refers to a peptide, polypeptide, or protein produced by recombinant DNA techniques; i.e., produced from cells transformed by an exogenous DNA construct encoding the desired peptide, polypeptide, or protein. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified.

The peptide/polypeptide/protein of this invention covers chemically modified versions. Examples of chemically modified peptide/protein include those subjected to conformational change, addition or deletion of a sugar chain, and those to which a compound such as polyethylene glycol has been bound. Once purified and tested by standard methods or according to the methods described in the examples below, the peptide/polypeptide/protein can be included in an immunogenic composition.

All of naturally occurring VSG proteins, genetic engineered versions, and chemically synthesized versions can be used to practice the invention disclosed therein. VSG peptides, polypeptides, or proteins obtained by recombinant DNA technology may have the same amino acid sequence as disclosed herein (e.g., SEQ ID NO: 1) or a functional equivalent thereof.

In the chimeric trypanosome VSG protein disclosed herein, the one or more heterologous polypeptide sequences can include sequences of an antigen or epitope. Any epitopes identified as capable of generating immune response in a subject may be inserted into positions within the sequence of the VSG protein, which facilitates surface display of the epitope. Insertion positions for the epitope of interest can be any position in the loops. Examples include insertion positions of the VSG sequence identified in FIG. 1 disclosed herein. As disclosed in the examples below, insertion positions can include one or more corresponding to residues 28, 144, 145, 153, 167, 203, 221, 227, 247, 249, 259, 262, 263, and 298, based on the sequence of VSG 221 a.k.a VSG 427-2 as shown in FIG. 1. In general, “insertion at position n” indicates that an insertion is between residues n and n+1. For example, an insertion at position 28 of VSG 427-2 means that an insertion is between residues 28 and 29 of the sequence shown in FIG. 1a.

It is to be noted that depending on the sequence of the insert, these locations may vary slightly (e.g. position 259 may be slightly better for one insert and position 263 better for another, though in both cases surface expression is broadly positive). Similarly, different insertion positions accommodate different lengths of insert, ranging from 1 to at least 50 amino acids, depending on the general location of the position (the loop of the VSG) and on the specific location of that insertion within each loop.

The epitope sequence to be inserted into the VSG can be of varying lengths. In certain embodiments, the sequence can be up to about 10, up to about 20 or up to about 50 amino acid residues in length. The sequence can be of a length sufficient to form a conformational epitope up to about 25 or about 50 amino acids in length. The epitope can be of any length, so long as the structure of the epitope is preserved and the integrity of the individual VSG count on the trypanosome is maintained.

### Recombinant Trypanosomes

This invention also provides recombinant trypanosomes and trypanosome-like particles. The recombinant trypanosomes and trypanosome-like particles of this invention display on their surfaces the above-described chimeric VSG protein having the heterologous polypeptide sequences and can be used in an immunogenic composition for generating antibodies against the heterologous sequences in a subject.

To produce the recombinant trypanosomes, one can transfect suitable trypanosomes cells with an expression vector encoding the above-descried chimeric VSG protein in the manner described in the examples below or by other standard techniques. T. brucei strains suitable in the present invention include, but are not limited to, Lister 427 strain. Lister 427 is a T. brucei strain that is one of the causative agents of N′ agana in cattle. The Lister 427 strain is not infectious to humans, where at least two serum factors, the lipoprotein apoL1 as well as haptoglobin act as trypanosome lytic factors, offering natural immunity against this organism. Taking advantage of the natural ability of the trypanosome to elicit highly specific antibody responses to the predominant coat, the VSG coat can be engineered to carry a chimeric coat composed of the predominant VSG and an internally expressed identical VSG, into which an epitope of interest can be inserted in-frame.

The aforementioned trypanosome-like particles are inactivated trypanosome cells. For example, trypanosome inactivation can be achieved by simple fixation (incubation for 1 to 30 min at room temperature in a fixation reagent, such as 1% formalin, followed by washes in PBS prior to injection). Other examples of the fixation reagent include chemical crosslinker, photoactivatable cross linkers, methanol, and glutaraldehyde. This procedure kills the parasite while leaving its proteins and DNA intact (though inactivated). Inactivation can also be achieved by more extensive processing that removes most cytoplasmic proteins as well as destroys DNA, leaving essentially an empty VSG coat (termed a “ghost”). To generate “ghost” trypanosomes, one can follow the protocol described in EXAMPLE 1 below or others known in that art. Immunized animals with engineered organisms (either live or formalin-fixed) can rapidly elicit high affinity anti-peptide antibodies. Such antibodies can be protective against infection with an organism displaying the same epitope.

### Conjugate Complex

The above-described trypanosome and VSG proteins can also be used as a part of immunogenic conjugate to elicit immune response to epitope or hapten, which by itself, elicit no or weak immune response.

The term “hapten” as used herein refers to a low-molecular weight organic compound that is not capable of eliciting an immune response by itself but will elicit an immune response once attached to a carrier molecule. The term “conjugate” refers to the product or complex of conjugation between one or more of (a) a core particle or carrier, such as the above-described VSG protein, trypanosome, or trypanosome-like particle, and one or more of (b) an organic molecule, hapten, antigen, or antigenic determinant as described herein, wherein the elements (a) and (b) are bound to each other.

In a preferred embodiment, a hapten of interest is attached to a core particle/carrier via a linker. To that end, the core particle/carrier contains, or is linked to (covalently or non-covalently), one or more first attachment sites; the hapten contains, or is otherwise linked to (covalently or non-covalently), one or more second attachment site(s). The second attachment site can be associated through at least one covalent or non-covalent bond to the first attachment site so as to form a hapten-carrier conjugate.

The first attachment site refers to an element of a core particle/carrier to which the second attachment site located on a hapten, antigen, or antigenic determinant may associate. The second attachment site refers to an element associated with the hapten to which the first attachment site may associate. Both the first attachment site and the second attachment site can be a protein, a polypeptide, a peptide, amino acid, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. In particular, the second attachment site can be any chemical moiety, e.g., an amine, an amide, a carboxyl, a sulflhydryl, hydroxyl, aldehyde, acylhalogenide, hydrazine, diazonium, or hydrazide, or further chemical moieties able to specifically react with the first attachment site. For a second attachment site, which is not naturally occurring within a hapten, the hapten can contain a linker which associates the hapten with the second attachment site.

In some embodiments, the first or second attachment site contains an antigen, an antibody or antibody fragment, biotin, avidin, strepavidin, a receptor, a receptor ligand, a ligand, a ligand-binding protein, an interacting leucine zipper polypeptide, an amino group, a chemical group reactive to an amino group; a carboxyl group, chemical group reactive to a carboxyl group, a sulfhydryl group, a chemical group reactive to a sulfhydryl group, or a combination thereof.

In certain embodiments, the hapten itself can contain one or more reactive functional group, to which the carrier can be attached directly, or via a linker, or via a matrix, or via a linker and a matrix. Preferably, the hapten is attached to the carrier protein via an amide or disulfide bond, which has the desirable property of stability. As the hapten-carrier conjugates of the invention can be used as vaccines, it is important that the conjugates are stable, to prolong the shelf life of the vaccine.

A hapten of the present invention can be a drug of abuse, including, but not limited to nitrophenyl, nicotine, methamphetamine, morphine, heroine, codeine, fentanyl; amphetamine, cocaine, methylenedioxymethamphetamine, methamphetamine, methylphenidate, cotinine, nornicotine, PCP, LSD, mescaline, psilocybin, tetrahydrocannabiflol, diazepam, desipramine, imipramine, nortriptyline, and the amitriptyline class of drugs, or a derivative thereof. As will be described in detail below, conjugates having one or more of these haptens are useful to prevent or treat addiction to drugs of abuse and the resultant diseases associated with drug addiction.

Methods for make conjugates are known in the art. For example, a single nicotine hapten can be directly attached to a carrier, with or without a linker, to each available amine group on a carrier. General methods for directly conjugating haptens to carrier proteins, using a homobifunctional or a heterobifunctional cross-linker are described, for example, by G. T. Hermanson in Bioconjugate Techniques, Academic Press (1996) and Dick and Beurret in Conjugate Vaccines. Contribu. Microbiol. Immunol., Karger, Basal (1989) vol. 10, 48-114. With direct conjugation using bifunctional crosslinkers, the molar ratio of hapten to protein is limited by the number of functional groups available on the protein for the specific conjugation chemistry. For example, with a carrier protein possessing n number of lysine moieties, there will be, theoretically, n+1 primary amines (including the terminal amino) available for reaction with the linker's carboxylic group. Thus, using this direct conjugation procedure the product will be limited to having n+1 amido bonds formed, i.e., a maximum of n+1 haptens attached. A VSG protein has a number of lysines. The skilled artisan will recognize that depending on the concentration of the reactants used to conjugate a hapten to the VSG carrier protein or a trypanosome, the ratio of hapten to carrier will vary. Also, within a given preparation of hapten-carrier conjugate, there will be variation in the hapten/carrier ratio of each individual conjugate.

The chimeric protein, related trypanosome, and conjugate discussed herein can be designed to contain any antigenic agent, antigen, immunogen, or epitope of interest. “Antigenic agent,” “antigen,” or “immunogen” means a substance that induces a specific immune response in a host animal. It can be a molecule containing one or more epitopes (either linear, conformational or both) that elicit an immunological response. The term “epitope” refers to basic element or smallest unit of recognition by an individual antibody, B-cell receptor, or T-cell receptor, and thus the particular domain, region or molecular structure to which said antibody or T-cell receptor binds. An antigen may consist of numerous epitopes while a hapten, typically, may possess few epitopes.

The system of chimeric protein, related trypanosome, and conjugate disclosed herein can be used as an antibody stimulating platform, to raise antibodies against any antigenic agent, antigen, immunogen, or epitope of interest. The antigen may contain a whole organism, killed, attenuated or live; a subunit or portion of an organism; a recombinant vector containing an insert with immunogenic properties; a piece or fragment of DNA capable of inducing an immune response upon presentation to a host animal; a protein, a polypeptide, a peptide, an epitope, a hapten, or any combination thereof. Alternately, the immunogen or antigen may contain a toxin or antitoxin.

The term “animal” includes all vertebrate animals including humans. It also includes an individual animal in all stages of development, including embryonic and fetal stages. In particular, the term “vertebrate animal” includes, but not limited to, humans, canines (e.g., dogs), felines (e.g., cats); equines (e.g., horses), bovines (e.g., cattle), porcine (e.g., pigs), as well as in avians. The term “avian” refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary.

In certain embodiments the epitope can come from a disease-causing microorganism or a parasite. For example, the epitope can be one from a virus, e.g., a flu invariant helix to elicit flu immunity, especially pan-flu immunity. Other influenza epitopes can be used to elicit immunity to various distinct influenza types or other epitopes of viral origin.

Examples of suitable antigens, epitopes, or immunogenic moieties include viral, bacterial, or parasitic antigens; inactivated viral, tumor-derived, protozoal, organism-derived, fungal, or bacterial antigens; toxoids, toxins; self-antigens; polysaccharides; lipids, fatty acids, proteins; glycoproteins; peptides; cellular vaccines; DNA vaccines; recombinant proteins; glycoproteins; and the like, for use in eliciting immune response to, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus and yellow fever, and Alzheimer's Disease. Especially, materials (such as recombinant proteins, glycoproteins, peptides, and haptens) that otherwise do not raise a strong immune response can be used in connection with the invention so as to elicit satisfactory response.

In some embodiments, the epitope can be a portion of a cancer antigen, such that antibodies against the epitope can raise specific anti-cancer immunity. This will be particularly interesting in situations where passive infusion of specific antibodies is known to be therapeutic (as is the case with neurofibromatosis, a childhood cancer), or where specific anti-tumor antibodies can bind to receptors present in certain cancer tissues (e.g. breast) and inhibit cancer growth (e.g. Trastuzumab/herceptin, broadly used in breast cancer treatment to block neu/her receptors).

The terms cancer antigen and tumor antigen are used interchangeably and refer to an antigen that is differentially expressed by cancer cells. Cancer antigens can be exploited to differentially target an immune response against cancer cells, and stimulate tumor-specific immune responses. Certain cancer antigens are encoded, though not necessarily expressed, by normal cells. Some of these antigens may be characterized as normally silent (i.e., not expressed) in normal cells, those that are expressed only at certain stages of differentiation, and those that are temporally expressed (e.g., embryonic and fetal antigens). Other cancer antigens can be encoded by mutant cellular genes such as, for example, oncogenes (e.g., activated ras oncogene), suppressor genes (e.g., mutant p53), or fusion proteins resulting from internal deletions or chromosomal translocations. Still other cancer antigens can be encoded by viral genes such as those carried by RNA and DNA tumor viruses.

Examples of tumor antigens include MAGE, MART-1/Melan-A, gp100, Dipeptidyl peptidase IV (DPPUV), adenosine deaminase-binding protein (ADAbp), cyclophilin b, Colorectal associated antigen (CRC)-C017-1A/GA733, Carcinoembryonic Antigen (CEA) and its antigenic epitopes CAP-1 and CAP-2, etv6, am11, Prostate Specific Antigen (PSA) and its antigenic epitopes PSA-1, PSA-2, and PSA-3, prostate-specific membrane antigen (PSMA), T-cell receptor/CD3-.zeta. chain, MAGE-family of tumor antigens (e.g., MAGE-A1 MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-3, SSX-4, SSX-5, SCP-1 and CT-7, and c-erbB-2.

Cancers or tumors and specific tumor antigens associated with such tumors (but not exclusively), include acute lymphoblastic leukemia (etv6, am11, cyclophilin b), B cell lymphoma (Ig-idiotype), glioma (E-cadherin, α-catenin, β-catenin, gamma-catenin, and p120ctn), bladder cancer (p21ras), biliary cancer (p21ras), breast cancer (MUC family, HER2/neu, c-erbB-2), cervical carcinoma (p53, p21ras), colon carcinoma (p21ras, HER2/neu, c-erbB-2, MUC family), colorectal cancer (Colorectal associated antigen (CRC)-CO17-1A/GA733, APC), choriocarcinoma (CEA), epithelial cell cancer (cyclophilin b), gastric cancer (HER2/neu, c-erbB-2, ga733 glycoprotein), hepatocellular cancer (α-fetoprotein), Hodgkins lymphoma (Imp-1, EBNA-1), lung cancer (CEA, MAGE-3, NY-ESO-1), lymphoid cell-derived leukemia (cyclophilin b), melanoma (p5 protein, gp75, oncofetal antigen, GM2 and GD2 gangliosides, Melan-A/MART-1, cdc27, MAGE-3, p21ras, gp100), myeloma (MUC family, p21ras), non-small cell lung carcinoma (HER2/neu, c-erbB-2), nasopharyngeal cancer (Imp-1, EBNA-1), ovarian cancer (MUC family, HER2/neu, c-erbB-2), prostate cancer (Prostate Specific Antigen (PSA) and its antigenic epitopes PSA-1, PSA-2, and PSA-3, PSMA, HER2/neu, c-erbB-2, ga733 glycoprotein), renal cancer (HER2/neu, c-erbB-2), squamous cell cancers of the cervix and esophagus (viral products such as human papilloma virus proteins), testicular cancer (NY-ESO-1), and T cell leukemia (HTLV-1 epitopes).

In another embodiment, an invention can be used to elicit immunity against prion disease, whereby the VSG protein is modified with an insertion of a sequence encoding the PrP helix. The invention may protect against Transmissible Spongiform Encephalopathies (TSEs, such as “mad cow disease” or scrapie in sheep), a disease of extensive veterinary interest, or can also be protective against primary related human diseases such as Alzheimer's Disease (AD) and other so-called conformational disorders as discussed in detail below. In yet another embodiment, the invention can be used to elicit immune responses to small molecule haptens that are drugs of abuse. Exemplary uses and procedures are also discussed in detail below.

### Compositions

The reagents described above can be used in a vaccine formulation to immunize an animal. Thus, this invention also provides an immunogenic or antigenic composition (e.g., a vaccine) that contains a pharmaceutically acceptable carrier and an effective amount of a chimeric protein, a recombinant trypanosome cell, a trypanosome-like particle, or a conjugate described above. The carriers used in the composition can be selected on the basis of the mode and route of administration, and standard pharmaceutical practice.

The term “immunogenic” refers to a capability of producing an immune response in a host animal against an antigen or antigens. This immune response forms the basis of the protective immunity elicited by a vaccine against a specific infectious organism. “Immune response” refers to a response elicited in an animal, which may refer to cellular immunity, humoral immunity or both.

The term “pharmaceutical composition” refers to the combination of an active agent (e.g., a chimeric protein, related trypanosome, and conjugate disclosed herein) with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

Examples of an adjuvant include a cholera toxin, Escherichia coli heat-labile enterotoxin, liposome, unmethylated DNA (CpG) or any other innate immune-stimulating complex. Various adjuvants that can be used to further increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

A vaccine formulation may be administered to a subject per se or in the form of a pharmaceutical or therapeutic composition. Pharmaceutical compositions containing an antigenic agent of the invention (the chimeric protein, recombinant trypanosome cell, trypanosome-like particle, or conjugate described above) and an adjuvant may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the antigens of the invention into preparations which can be used pharmaceutically. Proper formulation is dependent upon the route of administration chosen.

For injection, vaccine preparations may be formulated in aqueous solutions, preferably in physiologically compatible buffers such as Hanks' solution, Ringer's solution, phosphate buffered saline, or any other physiological saline buffer. The solution may contain formulatory agents such as suspending, stabilizing and/or dispersing agents. Alternatively, the chimeric protein, recombinant trypanosome cell, trypanosome-like particle, or conjugate described above may be in powder form for constitution with a suitable vehicle, e.g., sterile pyrogen-free water, before use.

An effective amount refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment.

The amount of a composition administered depends, for example, on the particular antigen in the composition, whether an adjuvant is co-administered with the antigen, the type of adjuvant co-administered, the mode and frequency of administration, and the desired effect (e.g., protection or treatment), as can be determined by one skilled in the art. Determination of an effective amount of the vaccine formulation for administration is well within the capabilities of those skilled in the art, especially in light of the detailed disclosure provided herein. An effective dose can be estimated initially from in vitro assays. For example, a dose can be formulated in animal models to achieve an induction of an immune response using techniques that are well known in the art. One having ordinary skill in the art could readily optimize administration to all animal species based on results described herein. Dosage amount and interval may be adjusted individually. For example, when used as a vaccine, the vaccine formulations of the invention may be administered in about 1 to 3 doses for a 1-36 week period. Preferably, 1 or 2 doses are administered, at intervals of about 3 weeks to about 4 months, and booster vaccinations may be given periodically thereafter. Alternative protocols may be appropriate for individual animals. A suitable dose is an amount of the vaccine formulation that, when administered as described above, is capable of raising an immune response in an immunized animal sufficient to protect the animal from an infection for at least 4 to 12 months. In general, the amount of the antigen present in a dose ranges from about 1 pg to about 100 mg per kg of host, typically from about 10 pg to about 1 mg, and preferably from about 100 pg to about 1 pg. Suitable dose range will vary with the route of injection and the size of the patient, but will typically range from about 0.1 mL to about 5 mL. Sera can be taken from the subject for testing the immune response or antibody production elicited by the composition against the antigen. Methods of assaying antibodies against a specific antigen are well known in the art. Additional boosters can be given as needed. By varying the amount of the composition and frequency of administration, the protocol can be optimized for eliciting a maximal production of the antibodies.

A composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils can be conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

### Therapeutic Vaccines Against Conformational Disorders

Several neurodegenerative diseases are characterized by the accumulation of a constitutively expressed protein in an abnormal, toxic conformation. The conversion from normal self-protein to an amyloidogenic, pathological conformer requires distinct structural changes, usually a switch from a predominantly alpha-helical structure to a structure enriched in beta-sheets, which is resistant to degradation.

For example, Alzheimer's disease, the most common cause of dementia, is a conformational disorder. The disease state in Alzheimer's disease is characterized by the conformational change of normal soluble amyloid-beta (A beta) peptide to a toxic oligomeric A beta. This conformational change is believed to give rise to a cascade of deleterious events, including hyperphosphorylation and accumulation of Tau protein, which lead to neuronal loss. Intriguingly, recent work has described a direct association between PrP^{C}, a host prion protein heavily implicated in the pathogenesis of transmissible spongiform encephalopathies, and A beta oligomers, with the former implicated as a natural pro death receptor for the latter.

Therapeutic approaches that ameliorate or prevent Alzheimer's disease currently do not exist, and disease outcome is invariably fatal. However, a number of recent reports have shown that immunization with A beta or Tau peptide can be beneficial in animal models of Alzheimer's disease and Tauopathies, respectively (Asuni et al., 2007, Journal of Neuroscience 27, 9115-9129 and Schenk et al., 1999 Nature 400, 173-177). Furthermore, a recent study demonstrated that anti-PrP immunization significantly ameliorated Alzheimer's progression in mouse models (Chung et al., 2010, BMC Neurosci 11, 130).

Despite positive results in animal models, active immunization with the A beta peptide proved unsuccessful in human trials due to the appearance of an inflammatory response mediated by T-cells. But overall, there is evidence that antibody mediated responses may be critical to the clearance of the pathogenic forms of these proteins, possibly by inhibiting the conformational switch of the normal to the pathogenic isoform.

Here, the instant invention provides a use of transgenic T. brucei as a display platform with which to generate antibody responses as well as long-term memory (without triggering T-cell responses and the resultant sequelae that have hampered clinical trials in the past). To begin, prion disease is used as a model system for conformational disorders, as it is far more manageable than murine models of Alzheimer's disease; and, the correlation between reduction of PrP-SC and disease prognosis is tight (White et al., 2003, Nature 422, 80-83).

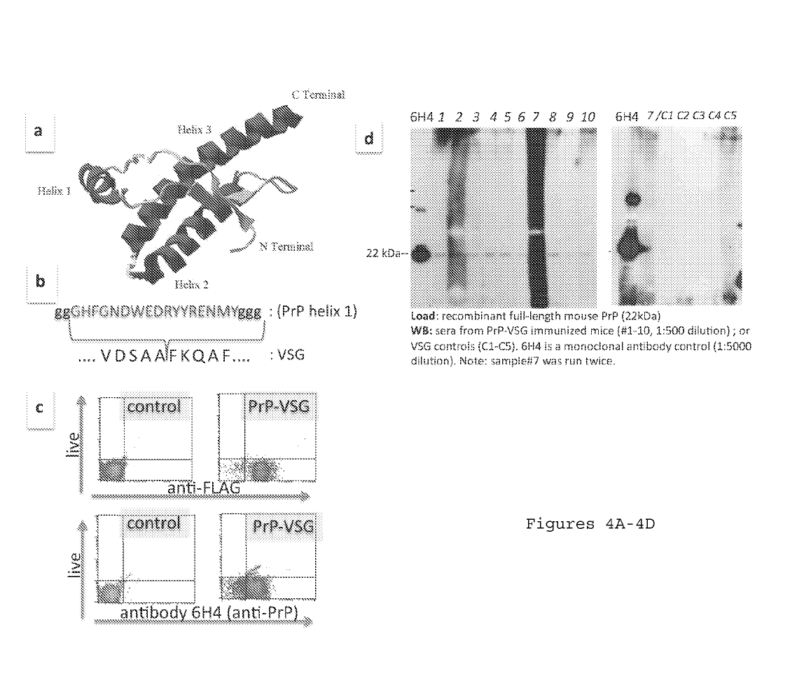

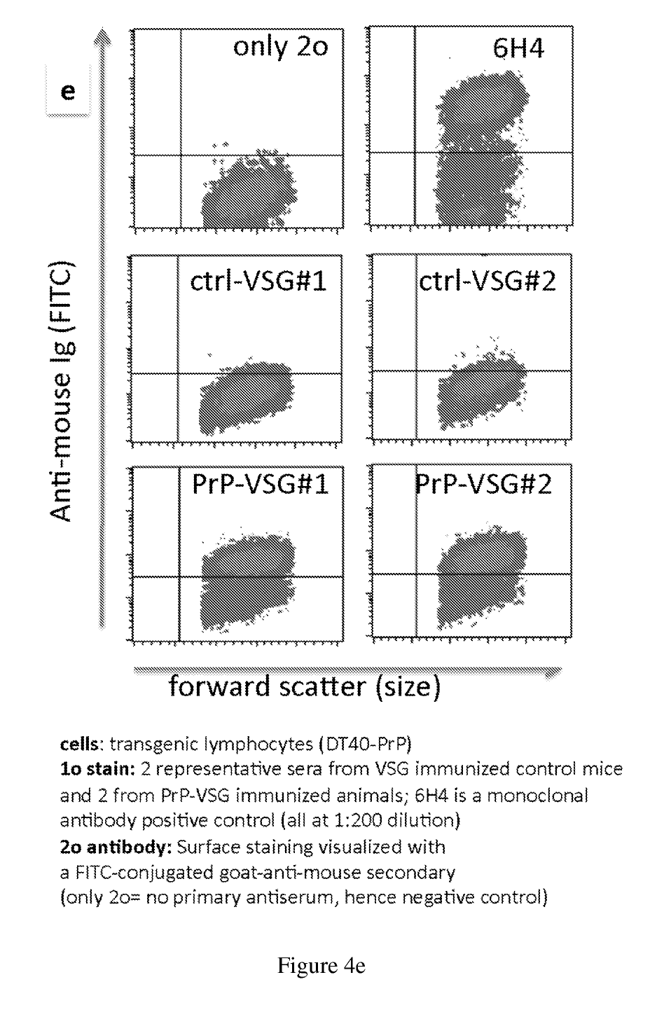

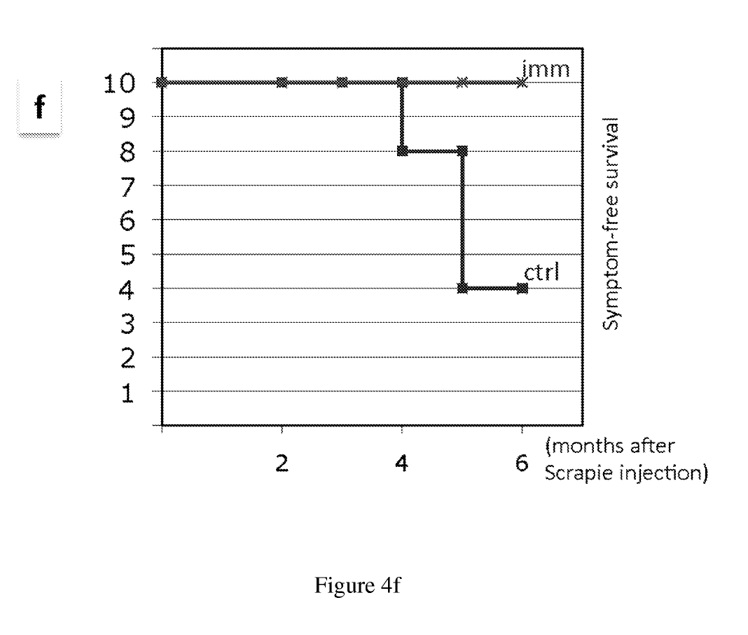

Thus far, transgenic VSGs displaying specific PrP mini-helices (PrP/VSG 427-2) were generated according to methods described above by the inventors or others under the inventors' direction. See FIG. 4. T. brucei cells expressing PrP/VSG 427-2 were also generated in the manner described above. These cells were fixed and injected into wild type C57BL/6 mice (with ˜10⁷/mouse). Subsequently the mice were boosted twice with the same composition. Based on preliminary data, it is expected that this immunization regimen will give rise to specific, neutralizing antibodies.

To assess the immunization regimen, one can collect and characterize antisera and clone antibodies elicited by the infection (e.g. by making hybridoma fusions) to understand how they interact with the inserted PrP peptide and the full-length prion protein. In addition, assays can be carried out to evaluate the cloned antibodies for protective and therapeutic potential. Interaction with the recombinant prion protein can be assessed by western blotting and ELISA; the therapeutic potential of the resultant anti-sera can be preliminarily assessed by FACS for their ability to bind extracellular surface bound PrP, which is a positive predictor of the value of these antibodies against prion diseases (Polymenidou et al., 2004, Proc Natl Acad Sci USA 101 Suppl 2, 14670-14676). Indeed, according to preliminary data, this immunization regiment has given rise to specific antibodies as assessed by Western blot (FIG. 4d). Based on their ability to bind PrP on the surface B cells (Polymenidoy et al, 2004, supra), these antibodies are also predicted to be protective (FIG. 4e).

One can also evaluate the possible therapeutic effect of VSG-PrP immunization in a murine model of prion diseases. Briefly, immunized mice have been challenged intraperitoneally with RML scrapie and subsequently monitored continuously for the appearance and development of symptoms (a battery of tests to assess their cognitive abilities performed on a weekly basis). Preliminary data show that immunized mice are protected from prion disease (that is, they do not show symptoms, or might show delayed symptoms, as compared to mice immunized with control trypanosomes that do not carry the prion epitope—FIG. 4f). To fully assess protection terminally ill mice can be sacrificed and accumulation of PrP^{Sc} in brain can be evaluated by western blotting and immunohistochemistry.

A similar approach can be used to models of Alzheimer's disease. Recent experiments have shown that infusion with anti-PrP antibodies resulted in the effective amelioration of Alzheimer's disease. To that end, one can immunize the proper animal models of Alzheimer's to recapitulate the “passive immunization” findings with the reagents already disclosed in FIG. 4 of the invention (Chung et al., 2010, BMC Neurosci 11, 130).

In addition, one can display both A beta and phosphorylated Tau peptides in the context of VSGs and immunize multiple models of Alzheimer's disease with the resultant transgenic trypanosomes either individually or concurrently. It has been proposed that the outcome of vaccination against Alzheimer's disease in animal models improves when both A beta and Tau are targeted at the same time. Concurrent immunization can be simply achieved by combining transgenic trypanosome inoculations, either in a single dose, or in alternate doses. It can also be achieved by engineering a trypanosome to express both A beta and Tau peptides using methods disclosed in this invention. See FIG. 4 and related description, which prove feasibility for expressing two distinct peptides (FLAG and PrP peptides) on trypanosome surfaces. In all these contexts, immunized mice can be monitored continuously for the appearance and development of Alzheimer's symptoms; and their cognitive abilities can be assessed using appropriate tests (e.g. water and Y maze). Furthermore, a small number of animals can be sacrificed at regular intervals to perform neuropathological and immunohistochemical analyses for neurodegenerative lesions in fixed tissue, and electrophysiological analyses on immobilized fresh tissue for LTP activity.

Finally, once this form of vaccination leads to targeting of normal isoforms of A beta and Tau, and the resultant toxicity (as was observed in clinical trials), one can accommodate the screening of peptide epitopes of similar conformation but using amino acid sequence that is not normally present in humans (as described by Wisniewski and colleagues, in Goni et al, PLoSONE, 2010).

The above-described procedures can be used to significantly increase antibody titers against the proteins in these conformational disorders and therefore, to contribute positively and directly to enhanced therapeutic approaches.

Generating Antibodies Against Small Molecules that are Drugs of Abuse

A slightly different type of therapeutic vaccination with fewer associated safety concerns has recently entered clinical development: immunization against drugs of abuse. Such vaccination aims to elicit specific antibodies to bind a drug in the bloodstream and block its uptake into the brain, thus blocking the psychotropic effects of the substance (and thus, its addictive potential). See, e.g., Chung et al. BMC Neuroscience 2010, 11:130, Freir et al. Nat Commun. 2011 Jun. 7; 2:336, Goñi et al. (2010) Immunomodulation Targeting Abnormal Protein Conformation Reduces Pathology in a Mouse Model of Alzheimer's Disease. PLoS ONE 5(10): Hicks et al. Mol Ther. 2011 March; 19(3):612-9; Chi Nature Medicine, vol. 17 No 2, February 2011; and Polymenidou et al., 2004, Proc Natl Acad Sci USA 101 Suppl 2, 14670-14676).

Vaccines against drugs of abuse do not aim at replacing the addictive substance or eliminating craving symptoms. In fact they should not be considered therapeutic, but rather, interventional, in the sense that they should help to overcome relapses that re-instate full addiction. Indeed, in animal models, vaccination against nicotine or morphine derivatives results in reduced levels of the drugs in the brain and ablates their addictive properties: addicted animals lose their drug-dependence after vaccination (LeSage et al., 2006, Psychopharmacology (Berl) 184, 409-416.) Simple nicotine vaccines are currently in phase III trials, and early reports suggest that in individuals where these have elicited high titers of specific antibodies, they have significantly ablated relapse (Hatsukami et al., 2005, Clin Pharmacol Ther 78, 456-467).

Drugs of abuse are small molecules, which on their own they are not capable of eliciting antibody responses but they must be conjugated to a carrier and then emulsified in an adjuvant such as alum (nicotine conjugated to KLH, a protein derived from a maritime organism, is one modality currently in phase III trials). Alternatively, synthetic vaccines that contain additional, immunogenic epitopes are also in testing. However, in all cases the conjugation ratio (number of haptens per molecule of carrier) though not reported, is bound to be very low (usually in the order of tens of haptens per carrier), compared to antigenic arrays composed of peptides (in the case of T. brucei that being up to 11 million haptens per VSG coat array). This is likely the reason why most small molecule vaccines are not very effective in eliciting antibody responses, and why they all fail in eliciting immunological memory.

As disclosed herein, one can develop a T. brucei based vaccine that can elicit strong and long lasting B cell responses and B cell memory to small molecules. To that end, one can begin by using nitrophenyl (NP), a common hapten for which antibody reagents are available, as a model “drug of abuse.” One can derivatize the surface of fixed T. brucei in ways that will be accepting of standard small molecule conjugation chemistry:

(i) by generating biotinylated surfaces either directly (through endogenous expression of bacterial biotin protein ligase BirA within transgenic T. brucei containing the 15 amino acid long biotin acceptor peptide (GLNDIFEAQKIEWHE; with the lysine being the acceptor moiety) also known as “Avitag”), or indirectly (by treating T. brucei containing the Avitag peptide with BirA in a test tube); NP directly conjugated to small peptide tags derived from avidin (Hiller et al. 1991, Biochem J 278 (Pt 2), 573-585), or to whole streptavidin, can then be arrayed on derivatized T. brucei;

(ii) by generating transgenic T. brucei containing small peptides derived from avidin that retain binding to biotin, and arraying on those directly biotinylated NP (the reverse of (i), above). Such a derivatized surface can accept from small peptides (e.g. biotin-PrP) to whole proteins as well;

(iii) by generating transgenic T. brucei containing peptides that can serve as protease-cleavage (TEV or 3c or similar) sites; cleavage of a fixed transgenic coat can liberate both amino- and carboxy terminal ends that can be derivatized using diverse chemistries to directly accept NP.

In all these cases, derivatized T. brucei surface expression can be verified using anti-NP antibodies. After injection, the generation of specific antisera can be determined by ELISA. All necessary reagents to NP are commercially available. Finally, the generation of memory can be evaluated with standard reagents, but in addition, it will be compared to NP conjugated to chicken gamma globulin (NP-CGG) emulsified in alum, a reagent that has been extensively used to assess memory, but which was recently shown to lead to poor long-term memory (Dogan et al. 2009, Nat Immunol 10, 1292-1299). Essentially, one can treat T. brucei as a supramolecular conjugation surface to vastly increase the number of small molecules that can be presented to the immune system, thus both leading to effective and reproducible antibody responses, and to memory.

Once NP-conjugated to T. brucei system has been geared towards achieving high anti-NP titers and memory, one can use this model system to develop T. brucei conjugates to nicotine, because it offers a case where general parameters of central importance to vaccine development have been extensively studied (e.g. the conjugation chemistry of nicotine to linker both with regard to the nature of the linker and with regard to its placement on nicotine—de Villiers et al. 2010, Vaccine 28, 2161-2168), and where a strong case can be made that increased titers (in comparison to nicotine-KLH) would make for a more efficient vaccine (LeSage et al., 2006, Psychopharmacology (Berl) 184, 409-416).

Finally, one can use a similar way to develop T. brucei conjugated with 6-succinyl morphine (6-SM). 6-SM has already been conjugated to BSA (and more recently to tetanus toxoid). It is capable of generating anti-morphine, anti-heroin, and anti-acetylmorphine antibodies in rats (thus capturing all the pharmacologically active compounds to which heroin is catabolized, Kinsey et al. 2009, Immunol Cell Biol 87, 309-314). Conjugation chemistry linking 6-SM to a carrier protein that leads to such antibodies is known in the art. See, e.g., Anton and Leff 2006, Vaccine 24, 3232-3240.

To assess the effectiveness of T. brucei conjugated to 6-SM to elicit antibody responses, various conjugates can be injected in mice and resulting antibody titers be evaluated by direct and competitive ELISA assays. One can inject conjugates in B cell “memory-indicator” mice (Dogan et al. 2009, Nat Immunol 10, 1292-1299) to assess the possibility that they can lead to B cell memory. Finally, it is important to determine whether the elicited antibodies can cross the blood brain barrier. This can be determined with a number of standard methods, including immunohistochemical studies to evaluate the presence and potential adverse effects of such antibodies in brain. One can also test for the effectiveness of anti-opiate vaccines as interventions toward curbing heroin or morphine recidivism. To explore the efficacy of active immunization with opiate-conjugates of T. brucei, one can utilize models of relapse in rats.

Overall, antibodies generated to a supramolecular array of 6-SM can function as “sponges” that remove opiates from the blood before they can cross to the brain, therefore curbing their reinforcing effects and functioning as an adjunct to other pharmacotherapies aimed at addition treatments.

### Example 1

### General Material and Methods

This example describes general materials and methods used in EXAMPLES 2-7 below.

### Plasmid Constructs

A DNA fragment containing the full-length VSG427-2 (also known as VSG221 or MITat1.2) (residues 1-476) was amplified by PCR from cDNA derived from Trypanosoma brucei (Lister 427). The full-length VSG427-2 was used as a PCR template to generate VSG427-2 variants. For over-expression of internally tagged VSG427-2 variants in T. brucei, (termed FLAG-VSG), sequences encoding tagged VSGs were cloned into the HindIII and BamHI sites of the pUB39 plasmid.

T. brucei Strains, Growth and Transfection

The bloodstream-form trypanosomes derived from the Lister 427 (VSG427-2 expressing) strain were cultured in HMI-9 and transfected according to the methods described in Wirtz et al., 1999 Mol Biochem Parasitol 99, 89-101.

### FACS Analysis

T. brucei were prepared and stained for FACS according to standard procedures. The reagents used for staining cells for FACS analysis were an anti-VSG221-FITC conjugated rabbit polyclonal antibody, the monoclonal ANTI-FLAG® M2-FITC conjugated antibody (SIGMA-ALDRICH Cat. number F4049), and the PE labeled monoclonal anti-HA (MILTENYI BIOTEC Cat. number 120-002-687).

### Parasitic Infection and Clearance of Mice

The transgenic bloodstream-form trypanosomes derived from the Lister 427-2 (MITat1.2; VSG 221) used in this study were introduced into standard C57BL/6 or Balb/c mice by intraperitoneal (i.p.) injection. Specifically, the mice were injected with live 2-3×10³ parasites, or 10⁷ formalin fixed cells, in 200 uL of HMI-9 using a 25G^{5/8} needle. Six days after infection, the mice were injected with 20 ug of G418, which has been shown to lyse the parasites and result in clearance (Murphy et al. 1993, Antimicrob Agents Chemother 37, 1167-70). Boosts were conducted 21 to 30 days after initial injection.

### ELISA

Ninety-six well microtiter plates were coated with 100 microliters of a PBS solution containing a recombinant Flag tagged protein (10 ng/microliter) overnight. The wells were washed three times with PBS-0.5% TWEEN 20 and then blocked for two hours with PBS-0.5% TWEEN 20-3% milk-5% sucrose. The plates were incubated with 100 microliters of various dilutions of serum in PBS-0.5% TWEEN 20-3% milk for two hours followed by three washes with PBS-0.5% TWEEN 20. The plates were then incubated with 100 microliters of PBS-0.5% TWEEN containing a 1:5000 dilution of anti-mouse IgGs peroxidase conjugated antibody (SOUTHERN BIOTECH) for one hour followed by three washes with PBS-0.5% TWEEN 20. The plates were developed by adding 100 microliters of ABTS chromogen substrate solution from INVITROGEN and the OD was determined at 405 nm with an ELISA reader.

### Trypanosome Inactivation

Inactivation was achieved by simple fixation (incubation for 30 min at room temperature in 1% formalin followed by washes in PBS prior to injection). This procedure kills the parasite while leaving its proteins and DNA intact (though inactivated). Inactivation was also achieved by more extensive processing that removed most cytoplasmid proteins as well as destroys DNA, leaving essentially an empty VSG coat (termed a “ghost”). To generate “ghost” trypanosomes, one can follow the protocol published by Bohme and Cross 2002, Mol Biochem Parasitol 99, 89-101. Briefly, 10 million cells were resuspended in 200 microliter of ice-cold water containing 0.1 mM TLCK (a serine protease inhibitor) and placed on ice for 5 minutes. After centrifugation at 3,000 g for 5 minutes, the supernatant was discarded and subjected to fixation.

### Example 2

### Structural Analysis of VSGs

In this example, structural analysis of VSGs was carried out to identify insertion sites for ectopic peptides.

More specifically, structural analysis of the N-terminal domain of Lister VSG 427-2 (VSG221 or MITat1.2) revealed a compact homodimeric structure. The fact that the loops are well defined in the structure as well as their resistance to proteolytic cleavage indicates that they contribute to the overall structural integrity and function of this molecule.

To assess whether insertions of a short peptide sequence such as the FLAG epitope (DYKDDDDK) within these structured loops would be tolerated, a series of internally tagged VSGs using VSG427-2 as a backbone (termed FLAG-VSGs; FIG. 1a) were designed. In conjunction, a series of internally tagged VSG427-2 constructs were made where portions of the VSG loop coding regions were deleted and replaced with short peptide sequences.

### Example 3

### Chimeric VSGs Expressing Exogenous FLAG Peptide were Tolerated by T. Brucei

T. brucei parasites require surface expression of the VSG in order to survive. To examine tolerance of T. brucei parasites to exogenous peptides, tagged VSGs from an internal (ectopic) location were expressed, while leaving the endogenous VSG intact. In so doing, issues of potential lethality (e.g. should the tagged protein hinder proper VSG assembly) were circumvented and an analysis of live trypanosomes which expressed FLAG-VSG, the endogenous VSG alone or a mixture of both could be carried out. 2-5 clones per construct were generated and tested for their expression in whole trypanosome extract by dot blot and FACS analysis to ascertain surface expression (FIG. 1b). It was found that all of the T. brucei clones transfected with constructs where the FLAG sequence was inserted within VSG loops were well expressed (FIG. 1b). Surprisingly, none of the clones transfected with constructs where the FLAG sequence replaced a portion of a VSG loop, was expressed, suggesting that these sequences are important to the structural integrity of the protein.

The above results were unexpected and non-obvious. Given the size of each VSG and the existence of 11 million copies per T. brucei coat, it has been calculated that the external surface of each trypanosome is filled to capacity with VSG. Therefore, it is surprising that one can display exogenous peptides on the surface of the coat, within exposed VSG loops. Structural information available for two distinct VSGs, whose primary sequence lacks homology but whose three-dimentional configuration is near-identical, underscores this point: VSG loops are structured and stable with an unexpected high resistance to proteolytic digestion.

### Example 4

### Chimeric VSGs Displayed Peptides with Variable Efficiencies Dependent on Insertion Location

Overall, chimeric clones generated in EXAMPLE 3 fell into three categories with regard to surface expression: (a) clones that displayed only VSG427-2, but not FLAG-VSG, on the surface (e.g. FIG. 1b, clones 2, 4, 8, and 9; FIG. 1c), (b) clones that displayed VSG427-2 as well as low levels of FLAG-VSG on the surface (which were further binned into clones expressing low levels of the chimeric FLAG-VSG vs. those containing a population with no surface expression and a subpopulation with low surface expression (FIG. 1b, clone 6) and finally clones that prominently displayed the FLAG-VSG chimera on the surface (FIG. 1B, clones 3, 5, and 7). It was found that certain locations within the exposed VSG loops on the surface of T. brucei were not only tolerant to peptide insertions, but also displayed exogenous peptides with high efficiency (for a complete list, see Tables 1 and 2 below). In the tables, FT refers to FLAG tag (amino acid sequence: dykddddk); HA refers to the hemagglutinin peptide tag (sequence: ypydvpdya). Throughout, amino acid sequences referring to the VSG are capitalized, with inserted sequences in lower case.

TABLE 1
Single Expression Constructs

| Construct | Insertion | Amino acid | surface |
|---|---|---|---|
| Name | location | sequence | expression |
| ins.1.FT | 28-FT-29 | VDSAAggdykddddkggEKGFK | Half positive |
| ins.3.FT | 84-FT-85 | EINHGggdykddddkggTNRAK | Negative |
| ins.4.FT | 144-FT-145 | QTKESggdykddddkggGTSGC | One log positive |
| ins.5.FT | 153-FT-154 | CMMDTggdykddddkggSGTNT | Half positive |
| ins.8.FT | 167-FT-168 | GGTIGggdykddddkggGVPCKL | Negative |
| ins.9.FT | 184-FT185 | PKRPAggdykddddkggATYLG | Negative |
| ins.10.FT | 203-FT-204 | QADAAggdykddddkggNNFHD | Two logs positive |
| ins.11.FT | 221-FT-222 | SGHNTggdykddddkggNGLGK | Two logs positive |
| ins.12.FT | 227-FT-228 | GLGKSggdykddddkggGQLSA | Three quarters |
| | | | positive |
| ins.13.FT | 247-FT-248 | VANSQggdykddddkggTAVTV | Half positive |
| ins.14.FT | 259-FT-260 | LDALQggdykddddkggEASGA | Two logs positive |
| ins.15.FT | 262-FT-263 | LQEASggdykddddkggGAAHQ | Two logs positive |
| ins.16.FT | 281-FT-282 | KALTGggdykddddkggAETAE | Negative |
| ins.17.FT | 291-FT-292 | FRNETggdykddddkggAGIAG | Negative |
| ins.18.FT | 298-FT-299 | IAGKTggdykddddkggGVTKL | Two logs positive |
| ins.20.FT | 328-FT-329 | KKYFSggdykddddkggGHENE | Negative |
| ins.21.FT | 353-FT-354 | QNLVGggdykddddkggDNQPT | Negative |
| HA1 | 28-HA-31 | VDSAAggypydvpdyaggFKQAF | Two logs positive |
| | (EK deleted) | | |
| HA3 | 145-HA-146 | QTKESggypydvpdyaggGTSGC | Two logs positive |
| HA12 | 153-HA-154 | CMMDTggypydvpdyaggSGTNT | Negative |
| HA8 | 167-HA-168 | GGTIGggypydvpdyaggGVPCK | One quarter |
| | | | positive |
| HA10 | 203-HA-204 | QADAAggypydvpdyaggNNFHD | Two logs positive |
| HA14 | 221-HA-222 | SGHNTggypydvpdyaggNGLGK | One quarter |
| | | | positive |
| HA19 | 227-HA-228 | GLGKSggypydvpdyaggGQLSA | Negative |
| HA25 | 259-HA-260 | LDALQggypydvpdyaggEASGA | Half positive |
| HA30 | 263-HA-264 | LQEASggypydvpdyaggGAAHQ | One log positive |

TABLE 2
Dual Expression Constructs

| Con- | Inser- | | Inser- | | HA ex- | FT ex- |
|---|---|---|---|---|---|---|
| struct | tion 1 | Amino acid 1 sequence | tion 2 | Amino acid 2 sequence | pression | pression |
| HA43 | 153- | CMMDTggdykddddkggSGTNT | 262- | LQEASggdykddddkggGAAHQ | N/A | Half |
| | FT-154 | | FT-263 | | | positive |
| HA46 | 221- | SGHNTggdykddddkggNGLGK | 262- | LQEASggdykddddkggGAAHQ | N/A | One log |
| | FT-222 | | FT-263 | | | positive |
| HA47 | 227- | GLGKSggdykddddkggGQLSA | 259- | LDALQggdykddddkggEASGA | N/A | One log |
| | FT-228 | | FT-260 | | | positive |
| HA48 | 227- | GLGKSggdykddddkggGQLSA | 262- | LQEASggdykddddkggGAAHQ | N/A | Three |
| | FT-228 | | FT-263 | | | quarters |
| | | | | | | positive |
| HA49 | 259- | LDALQggdykddddkggEASGA | 262- | LQEASggdykddddkggGAAHQ | N/A | One log |
| | FT-260 | | FT-263 | | | positive |
| HA15 | 221- | SGHNTggypydvpdyaggNGLGK | 153- | CMMDTggdykddddkggSGTNT | Half | Negative |
| | HA-222 | | FT-154 | | positive | |
| HA17 | 221- | SGHNTggypydvpdyaggNGLGK | 262- | LQEASggdykddddkggGAAHQ | Half | Small |
| | HA-222 | | FT-263 | | positive | fraction |
| | | | | | | positive |
| HA18 | 221- | SGHNTggypydvpdyaggNGLGK | 227- | GLGKSggdykddddkggGQLSA | Half | Quarter |
| | HA-222 | | FT-228 | | positive | positive |
| HA20 | 227- | GLGKSggypydvpdyaggGQLSA | 153- | CMMDTggdykddddkggSGTNT | Very | Very |
| | HA-228 | | FT-154 | | small | small |
| | | | | | fraction | fraction |
| HA21 | 227- | GLGKSggypydvpdyaggGQLSA | 221- | SGHNTggdykddddkggNGLGK | Half | Half |
| | HA-228 | | FT-222 | | positive | positive |
| HA22 | 227- | GLGKSggypydvpdyaggGQLSA | 259- | LDALQggdykddddkggEASGA | One log | Quarter |
| | HA-228 | | FT-260 | | positive | positive |
| HA23 | 227- | GLGKSggypydvpdyaggGQLSA | 262- | LQEASggdykddddkggGAAHQ | One log | Quarter |
| | HA-228 | | FT-263 | | positive | positive |
| HA26 | 259- | LDALQggypydvpdyaggEASGA | 153- | CMMDTggdykddddkggSGTNT | Negative | Half |
| | HA-260 | | FT-154 | | | positive |
| HA28 | 259- | LDALQggypydvpdyaggEASGA | 262- | LQEASggdykddddkggGAAHQ | Half | Negative |
| | HA-260 | | FT-263 | | positive | |
| HA31 | 263- | LQEASggypydvpdyaggGAAHQ | 153- | CMMDTggdykddddkggSGTNT | Very | Half |
| | HA-264 | | FT-154 | | small | positive |
| | | | | | amount | |
| | | | | | positive | |
| | In- | | | | | |
| | fluenza | | FLAG | | Surface | |
| Con- | Helix | | loca- | | ex- | FT ex- |
| struct | location | Influenza Helix sequence | tion | FLAG sequence | pression | pression |
| B3 | 153- | CMMDTgggstqnaidgitnkvnsviegggSGTNT | 263- | LQEASggdykddddkggGAAHQ | Yes | Yes |
| | B3-154 | | FT-264 | | | |
| | PrP | | | | | |
| | helix | | FLAG | | Surface | |
| Con- | (H1) | | loca- | | ex- | FT ex- |
| struct | location | PrP helix sequence | tion | FLAG sequence | pression | pression |
| PrP-H1 | 28- | VDSAAggghfgndwedryyrenmyggGFKQA | 263- | LQEASggdykddddkggGAAHQ | Yes | Yes |
| | H1-31 | | FT-264 | | | |
| | AID | | FLAG | | Surface | |
| Con- | peptide | | loca- | | ex- | FT ex- |
| struct | location | AID peptide sequence | tion | FLAG sequence | pression | pression |
| RD4 | 227- | NTNGLGKSggyisdwdldpggGQLSAA | 263- | LQEASggdykddddkggGAAHQ | Yes | Yes |
| | RD4-228 | | FT-264 | | | |
| RD164 | 227- | NTNGLGKSggntfvenrertfjaweggGQLSAA | 263- | LQEASggdykddddkggGAAHQ | Yes | Yes |
| | RD164- | | FT-264 | | | |
| | 228 | | | | | |

### Example 5

### Heterologous Epitopes Displayed on Surface of T. Brucei Elicited Specific, High Affinity Antibody Responses

To assess whether the humoral immune system could respond to the FLAG-VSG coat by producing anti-FLAG antibodies, a FLAG-VSG clone was injected into C57BL/6 mice (clone #3, displaying high levels of FLAG on the surface—FIG. 1b). The infection was confirmed by assessing the level of parasitemia through a tail nick five days after injection, and then drug cleared the trypanosomes from the bloodstream the next day. Small amounts of sera were then collected at several time points after clearance and the antibody affinity maturation response assessed by ELISA. As a comparison, non-transfected T. brucei (control) and FLAG peptide conjugated to KLH were also injected into mice.

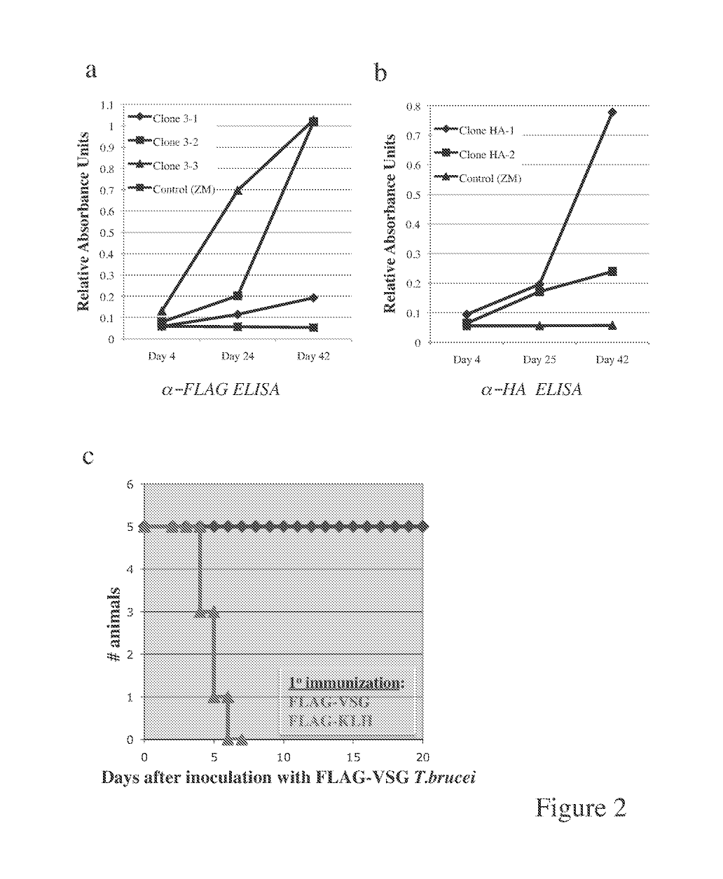

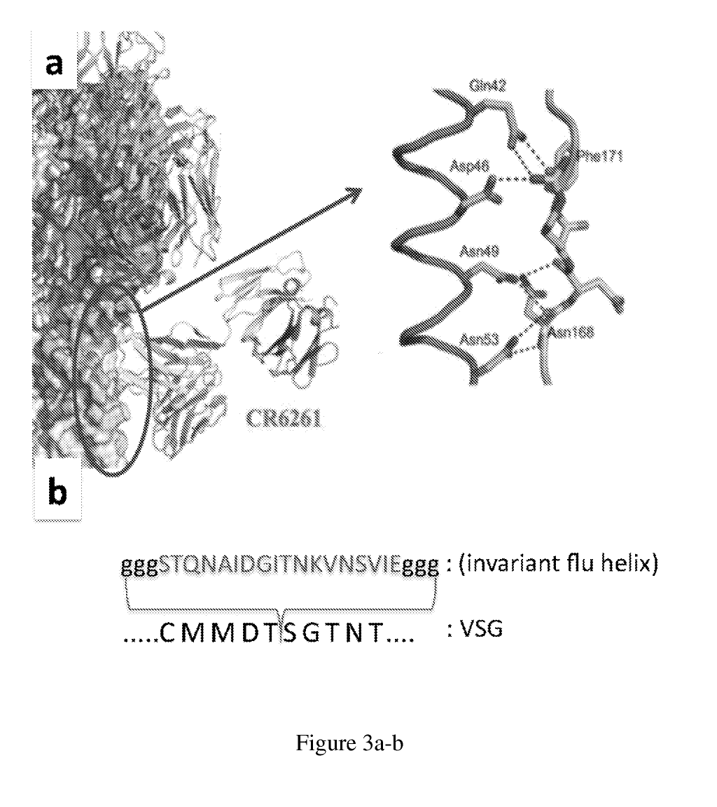

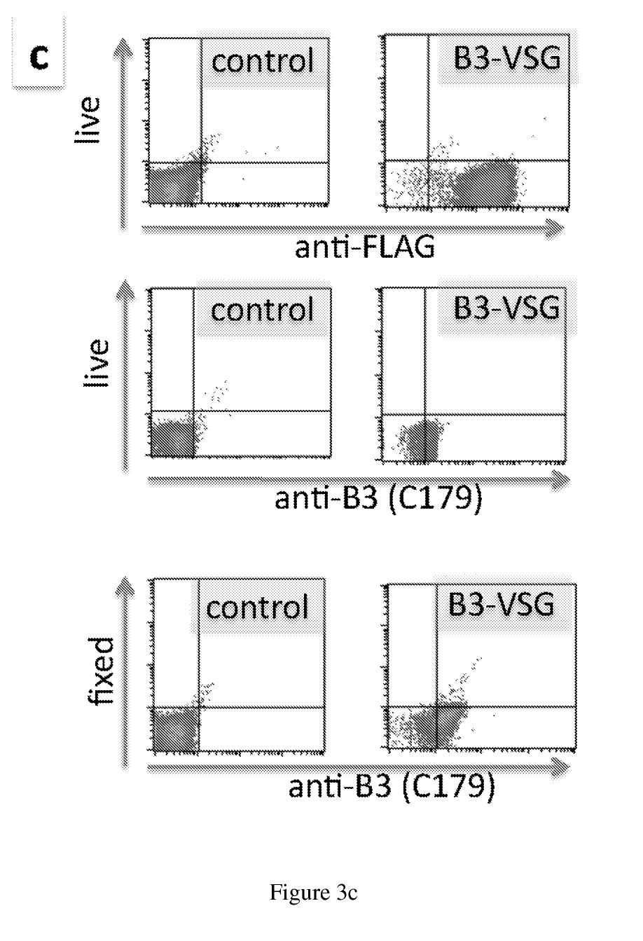

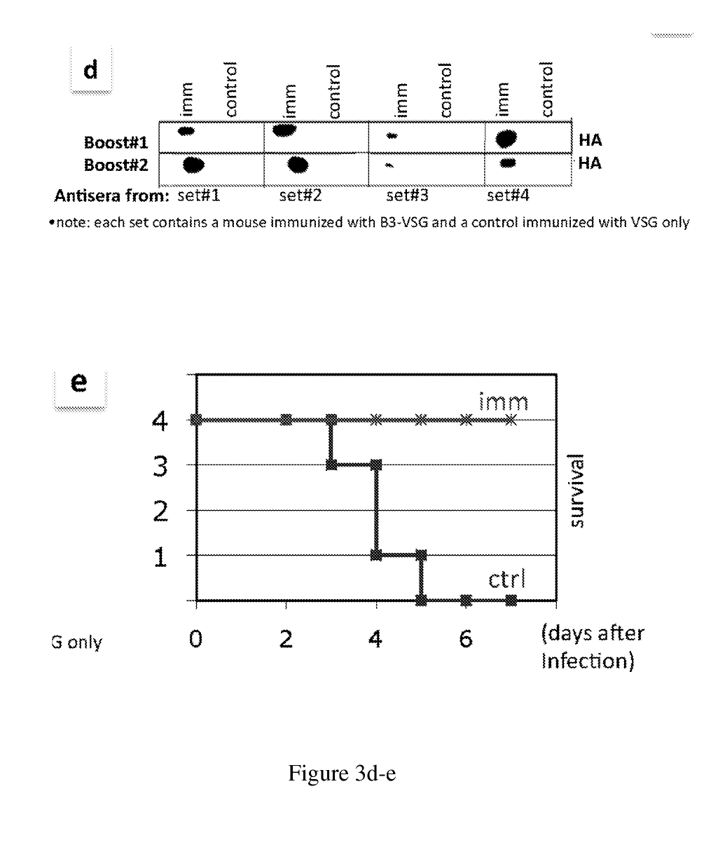

Surprisingly, it was found that infected mice mounted a robust and specific immune response against the FLAG peptide that was prominently displayed on the surface of the chimeric trypanosomes (FIG. 2b). Additionally, this antibody response could be amplified after a single boost, with day 42 representing 21 days post boost (FIG. 2a). These results demonstrate that chimeric trypanosomes can elicit a potent and specific antibody response against an exogenous peptide displayed on their surface coat.

### Example 6

### Fixed Chimeric Trypanosomes Elicited Antibody Responses

In EXAMPLE 5 above, live trypanosomes were injected into hosts. In order to address whether live trypanosomes are required for stimulation of an immune response against an epitope in tagged VSG, freshly formalin fixed trypanosomes were generated.

Trypanosomes can be fixed after a short treatment with formalin. However formalin fixation is likely to interfere with any epitope that contains lysines (such as FLAG). To assess whether fixed chimeric trypanosomes can elicit potent antibody responses such as those observed with the live organisms, trypanosomes were engineered to carry a chimeric/tagged VSG coat that displayed the HA peptide (YPYDVPDYA) on the surface (FIG. 1d), a peptide which does not contain lysine residues.

A number of clones expressing HA-VSG on the surface were obtained (FIG. 1d). Then, one of the HA-VSG clones and one of the FLAG-VSG clones were fixed. The fixed cells (10⁷ for each clone) were injected into C57BL/6 mice. It was found that these mice did not develop parasitemia. However, they did mount a strong antibody response against HA (FIG. 2b), though not against FLAG. The results demonstrate that fixed trypanosomes can elicit peptide-specific immune responses.

### Example 7

### FLAG-VSG Inoculation Protected Mice Against Re-Infection with Organism Displaying FLAG Epitope

As discussed herein, re-injection of a lethal dose of T. brucei with a certain coat into an animal previously inoculated against that coat led to complete clearance of the parasite. The result demonstrates that antibodies generated during the immune response are protective.

To determine whether this would also be the case for the antibody responses against the FLAG-VSG, FLAG-VSG-immunized C57BL/6 mouse (clone #3) and C57BL/6 mouse previously immunized with FLAG-KLH were re-inoculated with FLAG-VSG chimeric trypanosomes via intraperitoneal injection (100 FLAG-VSG trypanosomes per mouse). It was found that injection was lethal within ˜10 days, even if these mice were pre-immunized with FLAG-KLH. However, these animals survived infection with 10⁶ FLAG-VSG trypanosomes, if they had been previously inoculated with the same coat-carrying parasite (and subsequently cured through drug treatment). These results demonstrate a host response to a chimeric coat in the same manner as previously described for the unmanipulated parasite, in that it can be both specific and protective.

### Example 8

### Trypanosome Expressing a Protease Cleavage Site that Liberates VSG Ends as Hapten Conjugation Sites

One can engineer a protease cleavage site within one of the peptide insertion sites described. There are many different proteases that cleave specific recognition sequences. Such proteases include the Tobacco Etch Virus (TEV) protease and the human rhinovirus 3c protease. Upon cleavage of the VSG221 at the inserted protease cleavage site, a free amino terminal amine group and a free carboxy terminal carboxyl group will be generated. These two functional groups may be used as conjugation sites for the hapten of interest through commonly used chemical reactions for conjugation to these functional groups. Commonly used reactive group chemistries for hapten conjugation includes carbodiimides, N-Hydroxysuccinimide esters, and imidoesters among others.

One can either fix the trypanosome using cell and tissue fixing techniques that involves fixing agents such as formaldehyde and glutaraldehyde. After fixation, one can cleave the engineered VSG221 with the protease of interest in order to generate the reactive amine and carboxyl groups. After generation of these reactive groups one can perform the hapten conjugation reactions.

One can also create “ghost” trypanosomes by the methods described above. After the “ghost” is created on can cleave the VSG with the protease of interest in order to generate the reactive amine and carboxyl groups. After generation of these reactive groups one can perform the hapten conjugation reactions. One can then use as is or one can fix the cell using techniques that involves fixing agents such as formaldehyde and glutaraldehyde.

### Example 9

### Trypanosome VSG Expressing AID Protein

In this example, chimeric protein of VSG and AID was generated. AID is a protein of significant biomedical interest but one against which antibody generation has been extremely difficult, due to issues of immune tolerance. Indeed, mouse and human versions of AID are near-identical and are recognized as “self.” This “tolerance to self” problem, which is often insurmountable, has been routinely encountered when attempting to generate antibodies against tumour—i.e. “self” antigens).

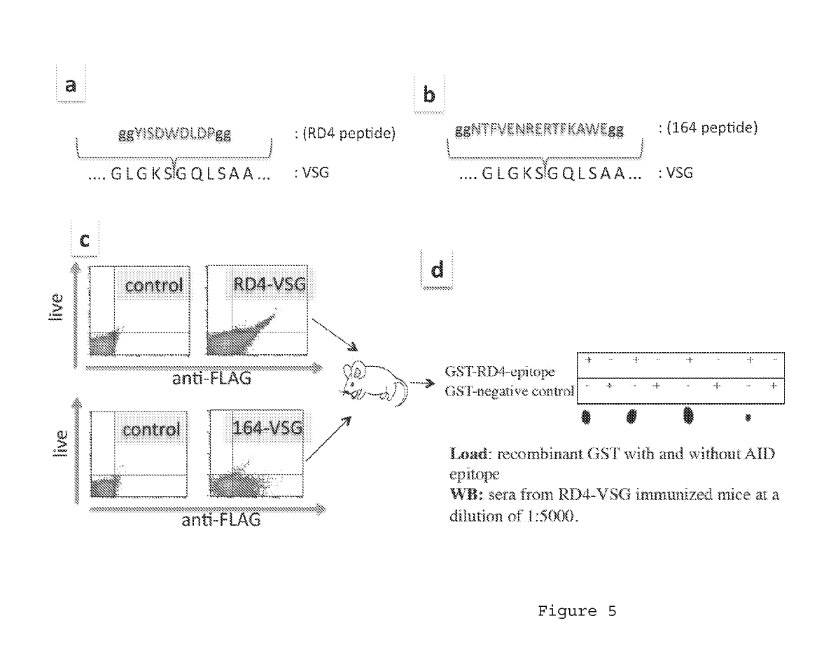

Here, two AID epitopes (named RD4 and 164) were used toward the generation of robust anti-AID antibodies that can bypass tolerance. As shown in FIG. 5a-c, epitopes of RD4 and 164 peptides were successfully inserted into VSG to generate chimeric proteins. The related FACS analysis results are shown in FIG. 5c). Recombinant trypanosomes expressing the chimeric proteins were also generated in the manner described above. The chimeric proteins and recombinant trypanosomes were administered to mice. It was found that both the chimeric proteins and recombinant trypanosomes elicited antibodies to the engineered epitopes in mice. The related Western blot analysis is shown in FIG. 5d.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein in their entireties.

1. A chimeric protein comprising (i) one or more alpha helices or beta-strands of a Trypanosoma brucei variable surface glycoprotein (VSG protein), and (ii) a first heterologous polypeptide sequence.

2. The chimeric protein of claim 1, wherein the chimeric protein further comprises a second heterologous polypeptide sequence.

3. The chimeric protein of claim 2, wherein the first or second heterologous polypeptide sequence is 1-50 amino acids in length.

4. The chimeric protein of claim 3, wherein the first or second heterologous polypeptide sequence is 2-50 amino acids in length.

5. The chimeric protein of claim 2, wherein the first and second heterologous polypeptide sequences are the same.

6. The chimeric protein of claim 2, wherein the first and second heterologous polypeptide sequences are different.

7. The chimeric protein of claim 1, wherein the first heterologous polypeptide sequence contains an epitope of a protein selected from the group consisting of a pathogen protein, a tumor antigen, an amyloid beta, a Tau peptide, and a prion protein (PrP^{C}).

8. The chimeric protein of claim 1, wherein the first heterologous polypeptide sequence contains SEQ ID NO: 2 or 3.

9. The chimeric protein of claim 1, wherein the VSG protein contains the sequence of SEQ ID NO: 1.

10. The chimeric protein of claim 1, wherein the chimeric protein contains a mutant version of SEQ ID NO: 1 where the first or second heterologous polypeptide sequence is inserted at one or more of positions 28, 144, 145, 153, 167, 203, 221, 227, 247, 249, 259, 262, 263, and 298.

11. A nucleic acid encoding the chimeric protein of claim 1.

12. A vector comprising the nucleic acid of claim 11.

13. A host cell comprising the nucleic acid of claim 12.

14. A recombinant Trypanosoma brucei comprising the chimeric protein of claim 1.

15. A conjugate comprising a Trypanosoma brucei variable surface glycoprotein (VSG protein) and a heterologous moiety, wherein the heterologous moiety is conjugated to the VSG protein.

16. The conjugate of claim 15, wherein the heterologous moiety is a polypeptide or a small molecule hapten.

17. The conjugate of claim 16, wherein the polypeptide contains an epitope of a protein selected from the group consisting of a pathogen protein, a tumor antigen, an amyloid beta, a Tau peptide, and a prion protein (PrP^{C}).

18. The conjugate of claim 16, wherein the small molecule hapten is one selected from the group consisting of nitrophenyl, nicotine, methamphetamine, morphine, and heroine, or a derivative thereof.

19. An immunogenic composition comprising the chimeric protein of claim 1 and a pharmaceutically acceptable carrier.

20. An immunogenic composition comprising the recombinant Trypanosoma brucei of claim 14 and a pharmaceutically acceptable carrier.

21. An immunogenic composition comprising the conjugate of claim 15 and a pharmaceutically acceptable carrier.

22. A method of producing antibodies that recognize a polypeptide in a subject, comprising administering to the subject the immunogenic composition of claim 1.

23. A method of eliciting an antigen-specific immune response in a subject, comprising administering to a subject in need thereof the immunogenic composition of claim 1.

24. (canceled)

25. (canceled)


SEQUENCE LISTING (3 sequences)

SEQ ID NO: 1 - protein, 363 aa, Artificial
  N-terminal domain of VSG427-2
AAEKGFKQAFWQPLCQVSEELDDQPKGALFTLQAAASKIQKMRDAALRASIYAEINHGTNRAKAAVIVANHYAMKADSGLEALKQTLSSQEVTATATASYLKGRIDEYLNLLLQTKESGTSGCMMDTSGTNTVTKAGGTIGGVPCKLQLSPIQPKRPAATYLGKAGYVGLTRQADAANNFHDNDAECRLASGHNTNGLGKSGQLSAAVTMAAGYVTVANSQTAVTVQALDALQEASGAAHQPWIDAWKAKKALTGAETAEFRNETAGIAGKTGVTKLVEEALLKKKDSEASEIQTELKKYFSGHENEQWTAIEKLISEQPVAQNLVGDNQPTKLGELEGNAKLTTILAYYRMETAGKFEVLTL

SEQ ID NO: 2 - protein, 8 aa, Artificial
  FLAG epitope
DYKDDDDK

SEQ ID NO: 3 - protein, 9 aa, Artificial
  HA epitope
YPYDVPDYA